(19) United States
(12) Patent Application Publication
(10) Pub. No.: US 2025/0354975 A1
(43) Pub. Date: Nov. 20, 2025

(54) SYSTEM FOR ANALYZING PLATELET RICH PLASMA

(71) Applicant: Patrick Pennie, Fort Myers, FL (US)

(72) Inventor: Patrick Pennie, Fort Myers, FL (US)

(21) Appl. No.: 19/204,893

(22) Filed: May 12, 2025

Related U.S. Application Data
(60) Provisional application No. 63/647,789, filed on May 15, 2024.

(51) Int. Cl. G01N 33/49; G01N 15/01; G01N 15/10; G01N 15/14; G01N 15/1429

(52) U.S. Cl. CPC G01N 33/491; G01N 15/1431; G01N 2015/018; G01N 2015/1006; G01N 2015/1402; G01N 2015/1488

(57) ABSTRACT

A PRP analyzing system is constructed and programmed to determine a baseline complete blood count of a whole blood sample and a blood count of PRP derived from the sample. The system compares the baseline and PRP blood counts and calculates qualitative PRP metrics such as constituent percentage yield, concentration and deliverable of PRP constituents. This provides immediate, understandable and compelling indication of the likely performance and effectiveness of the PRP in medical applications.

### FIELD OF THE INVENTION

This invention relates to a system for analyzing platelet rich plasma (PRP) and more particularly to a system wherein the measured blood counts of PRP derived from a blood sample are compared against the complete blood count measured for that blood sample to determine performance qualities of the PRP such as yields, deliverable quantities and concentrations of platelets and other constituents of the PRP.

### BACKGROUND OF THE INVENTION

A complete blood count (CBC) is a common medical test used to detect and diagnose infections, blood diseases, anemia, immune system disorders and other medical conditions. Specifically, the test measures CBC levels of various constituent elements and features of a blood sample including, but not limited to, white blood cells (WBC), red blood cells (RBC), hemoglobin, hematocrit, platelets, neutrophils, monocytes and plasma.

Platelet rich plasma has conventionally been used to treat clotting disorders. PRP has also been used widely in surgical, dental, veterinary and other medical applications for wound care and tissue regeneration. PRP is typically derived from a whole blood sample using one of a number of available systems and devices. Improved medical results are usually achieved when increased amounts of platelets are exhibited by the PRP. By the same token, increased levels of white blood cells, neutrophils and monocytes typically indicate an improved ability of the derived PRP to combat infection. Accordingly, obtaining a high quality PRP containing greater yields of platelets and other beneficial constituents is highly desirable.

Hematological or blood analyzers are conventionally known for measuring both complete blood counts (CBC) and PRP counts. As indicated above, the quality and resultant effectiveness of the derived PRP typically depends upon the percentage yields of the desirable blood constituents (e.g., platelets, monocytes, neutrophils, white blood cells) present in the derived PRP. The concentration and deliverable quantity of derived platelets also presents a good measure of the PRP's likely performance in a medical application. Currently, in order for the physician or other medical professional to determine the effective yield and concentration of the individual constituent blood components, he or she must perform tedious and time-consuming calculations independently of the analyzer. Such calculations are also required to determine the deliverable level of platelets available in the derived PRP. To date, no blood analyzers have been developed that perform these calculations and determine the performance quality of the PRP sample and its resultant effectiveness in medical applications.

### SUMMARY OF THE INVENTION

It is therefore an object of the present invention to provide a system for analyzing platelet rich plasma (PRP) wherein a blood analyzer is programmed to compare PRP counts against the complete blood count of a blood sample from which the PRP was derived in order to provide an automatic indication of PRP quality and likely performance without requiring tedious and time-consuming calculations independent of the analyzer.

It is a further object of the present invention to provide a platelet rich plasma (PRP) analysis system that quickly, conveniently and accurately measures and displays the percentage yields, concentrations and deliverable quantities of platelets and other medically beneficial constituent elements of PRP derived from a whole blood sample and which therefore reliably reflects the likely performance of the PRP and effectiveness of the equipment and process used to derive the PRP.

It is a further object of the present invention to provide a system wherein measurements of the complete blood count (CBC) of a whole blood sample and the count of a derived PRP are integrated into a single apparatus that further includes the ability to compare the PRP counts against the CBC to determine the yield, concentration and deliverable quantities of one or more constituent elements of the PRP and therefore the quality and likely effectiveness of the PRP.

It is a further object of the present invention to provide a PRP analysis system that automatically and instantaneously calculates and displays the deliverable platelet level for a derived PRP.

This invention features a system for analyzing constituent elements of PRP derived from a whole blood sample. The system includes means for determining a complete blood count (CBC) for the whole blood sample and means for determining a PRP count for PRP derived from the sample. Means are provided for displaying the determined CBC and PRP counts and calculator means are provided for receiving electronic inputs from the means for determining the CBC counts and PRP counts. The means for determining and calculator means also receive volume inputs related to the whole blood sample and PRP. The calculator means are actuated electronically to compare the determined CBC count with the determined PRP count and calculate one or more qualities of the PRP with respect to the blood sample. Such qualities include the percentage yield of individual blood constituents, the concentration of individual blood constituents and the deliverable level of individual blood constituents in the PRP. The display means may include a user interactive interface such as an electronic touch screen operably connected to an output from the calculator for displaying the performance and quality results relating to derived PRP. The interface may also be used to input the whole blood and derived PRP volumes into the means for determining and calculator means. The interface, means for determining and calculator means may be programmed to enable the user to direct one or more microprocessors or other electronic components defining the means for determining and calculator means to perform their respective calculations. In preferred embodiments, the calculator means and interface may be programmed to calculate and display the actual qualitative test data for platelets or other constituent components of a specific PRP dosage introduced at a patient injection site.

The system is integrated into and operated in a hematology or blood analyzer that includes hardware such as microprocessors, sensors and other electronics that will be understood to persons skilled in the art. Initially, a whole blood sample is prepared and a small amount of the sample is introduced into the analyzer. A user interface allows the volume of the sample and a sample identification to be input. The analyzer is then operated to determine and generate a complete blood count that may be displayed on the display screen of the analyzer. PRP is then derived from the blood sample and a small amount is introduced into the analyzer, which determines a PRP count for the derived PRP. That count can also be optionally displayed. Once again, the volume of the derived PRP is input via the user interface. The calculator means may be accessed via a touch screen display to automatically perform comparative calculations that produce the percentage yields, concentrations and deliverables of the PRP sample based upon the respective measured counts and volumes of the whole blood sample and derived PRP. The calculator and display means may respectively calculate and display qualitative test data such as delivered number of platelets for a selected PRP dosage volume at a patient injection site. This better enables the physician to assess the efficacy of that dosage in treating the patient.

### BRIEF DESCRIPTION OF THE DRAWINGS

Other objects, features and advantages will occur from the following description of a preferred embodiment and the accompanying drawings, in which:

FIG. 1 is a schematic view of the platelet rich plasma analysis system;

FIG. 2 is a flow chart depicting operation of the system;

FIG. 3 is an elevational view of a preferred home display screen used for this system, which screen provides electronic buttons for selectively testing a baseline whole blood sample and PRP derived from that sample. The home screen also features an archive button for selectively accessing a stored whole blood sample's CBC test results;

FIG. 4 is an elevational view of a display screen that allows the user to enter a test identification number for a particular baseline blood sample being tested;

FIG. 5 is an elevational view of the display screen depicting the use of an electronic keyboard to identify the baseline blood sample test;

FIG. 6 is an elevational view of the screen display being used to enter the volume of the baseline blood sample;

FIG. 7 is a front elevational view of the display screen with the baseline CBC sample test in progress;

FIG. 8 is a front elevational view of the display screen depicting the baseline test results;

FIG. 9 is a front elevational view of the display screen specifically referencing the PRP count test button;

FIG. 10 is a front elevational view of the display screen in the PRP test mode and specifically listing archive baseline tests; the baseline test corresponding to the derived PRP to be tested is specifically indicated;

FIG. 11 is a front elevational view of the display screen instructing the user to insert the PRP into the analyzer;

FIG. 12 is a front elevational view of the display screen providing the user with an option to designate the volume of PRP being tested;

FIG. 13 is front elevational view of the display screen listing the PRP test results;

FIG. 14 is a front elevational view of the display screen listing the calculated performance levels for the PRP including the yields of individual constituent components, the platelet concentration ratio and the deliverable platelets in the derived PRP; and

FIG. 15 is a front elevational view of the user interactive display screen listing calculated test data, i.e. number of delivered platelets, in a selected dosage of injected PRP.

### DETAILED DESCRIPTION OF INVENTION

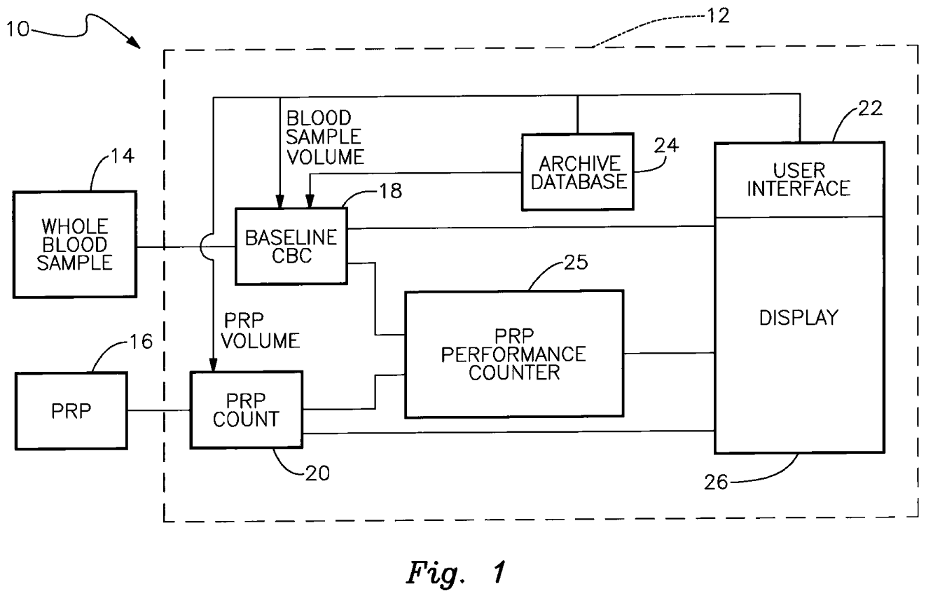

There is shown in FIG. 1 a platelet rich plasma (PRP) analyzing system 10 for determining the performance and quality characteristics of PRP derived from, and in comparison to the complete blood count (CBC) of a baseline blood sample from which the PRP was derived. System 10 is integrated into and used in conjunction with a hematologic or blood analyzer 12 of the type conventionally used for determining the complete blood count of a whole blood sample 14 and/or the blood count of PRP 16 derived from that sample. Analyzer 12 may comprise, for example, a Regenistat™ platelet analyzer distributed by Emcyte Corporation or an alternative apparatus for analyzing blood counts.

Analyzer apparatus 12 includes known means (e.g., hardware such as sensors, microprocessors and other electronic components/circuitry) for determining the complete blood count (CBC) and the PRP count of whole blood 14 and PRP 16, respectively. For example, a microprocessor 18 or analogous means is used to determine a baseline CBC for blood sample 14. By the same token, a microprocessor 20 or analogous circuitry may be employed to determine the blood count of derived PRP 16. These microprocessors are arranged and programmed to calculate these respective blood counts in a manner that will be known to persons skilled in the art. In alternative embodiments, a single microprocessor may be programmed to determine both the CBC and PRP count. A user interface 22, which may be incorporated into a display screen 26 of analyzer 12, is employed to input the volume of both the whole blood sample and the derived PRP into the respective microprocessors. Alternatively, such volumes may be input directly into a PRP performance calculator 25 which is described more fully below. The baseline sample and PRP volumes are especially important for calculations performed by calculator 25 comparing the baseline CBC count and the PRP count to determine the performance and quality of the derived PRP in accordance with this invention. This calculation and additional operations, which were previously absent from the prior art are described more fully below. As further shown in FIG. 1, user interface 22 may also be employed to access an archive database 24 of various CBC results from a plurality of tested whole blood samples. As clearly illustrated below, a selected alternative baseline CBC may be compared against a measured PRP count corresponding to that alternative baseline CBC.

A critical component of this invention is the PRP performance calculator 25, which may comprise a microprocessor or analogous electronic component(s) programmed to process signals representative of the CBC and PRP counts and respective volumes and calculate qualitative measures of the derived PRP. In particular, calculator 25 receives input signals from baseline CBC component 18 and PRP count component 20 and compares those counts and respective input volumes of the tested whole blood sample and PRP to calculate the quality or performance of the PRP with regard to the baseline blood sample. The qualities determined by calculator 25 include, for example, the percentage of constituent components such as platelets, white blood cells, red blood cells, neutrophils and monocytes derived by the PRP from the baseline blood sample. Concentrations of the constituents, as well as levels of deliverable platelets and other deliverable constituents present in the PRP may also be determined by calculator 25. It should be understood that means for determining 18 and 20 and calculator 25 may comprise and be programmed on a single microprocessor or multiple microprocessors or analogous electronic components within the scope of this invention.

Each of baseline CBC component 18, PRP count component 20 and PRP performance calculator 25 is operably connected to an electronic display 26 that is operably integrated with the analyzer apparatus 12. Operation of that display is disclosed more fully below.

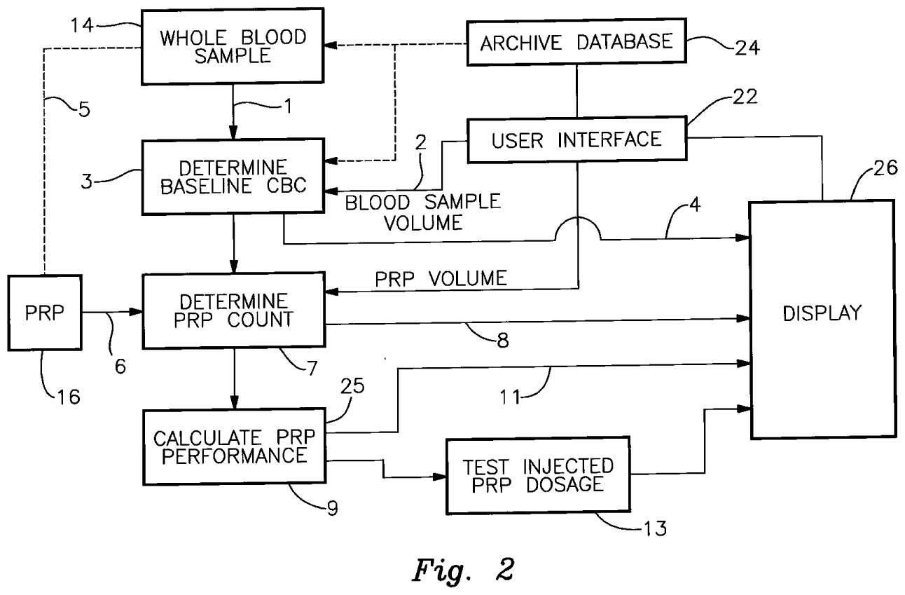

FIG. 2 depicts a flow chart reflecting the program used by PRP analyzing system 10. A baseline whole blood sample 14 is collected, typically in a syringe, and prepared for testing. A small amount of blood sample 14 is then introduced into the analyzer apparatus for testing, Step 1. The user then enters the volume of the whole blood sample into the analyzer, Step 2, using the user interface 22. The analyzer then determines a baseline CBC for the whole blood sample, Step 3, and delivers that baseline CBC to display 26, Step 4.

PRP 16 is derived from whole blood sample 14 using equipment and procedures disclosed, for example, in U.S. Pat. Nos. 6,835,353, 7,976,796, 10,537,888, 10,987,762, Step 5. A small amount of the derived PRP is then introduced into the analyzer apparatus, Step 6, and the analyzer operates to determine a PRP count, Step 7. The PRP count is then transmitted to and displayed on display screen 26, step 8.

After the CBC and PRP counts have been determined, these results are processed in accordance with this invention by the analyzer's integral PRP performance calculator 25, step 9, to automatically derive the performance and quality results of the PRP with respect to the baseline blood sample. These results, which are transmitted to and display 26, step 10, can include the percentage yields of constituents in the PRP relative to the constituents in the baseline blood sample, as well as concentrations and deliverable quantities of platelets and other valuable constituent components of the blood sample. More specifically, those results may comprise respective yields of white blood count, red blood count, neutrophils, monocytes, platelets, as well as platelet concentrations and total platelets deliverable. These results are described more fully below.

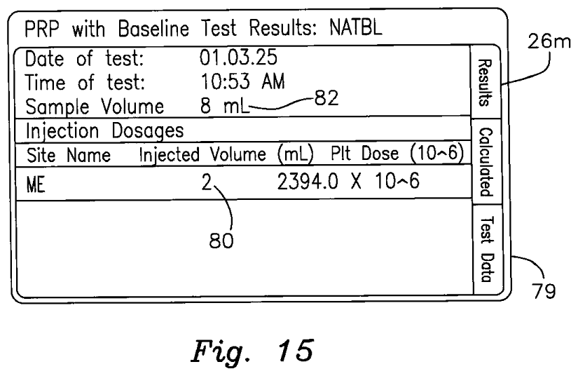

Calculator 25 may also be programmed to calculate the actual number of platelets or other constituent components that will actually be delivered in a selected injected dosage volume of the derived PRP. That data is likewise directed either automatically or by using interface 22 to display 26, where it is displayed as described more fully below in connection with FIG. 15.

FIG. 2 further shows archived database 24, which may store the results of additional tests performed on whole blood sample 14. The user may select one of those additional tests as an alternative baseline to compare against corresponding PRP counts using the foregoing program.

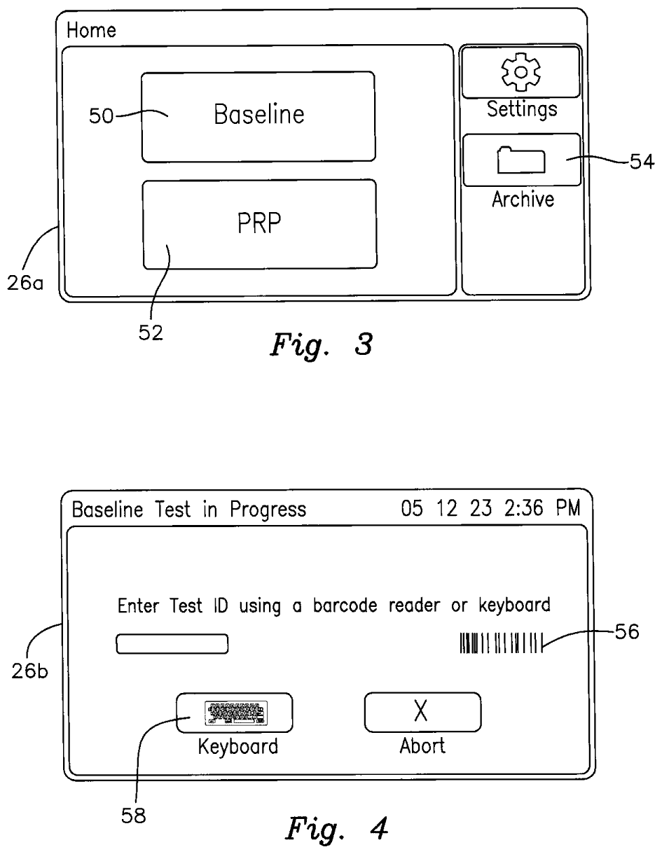

FIGS. 3-14 disclose successive representative views of screen display 26 (respectively designated by successive lower-case letters) illustrating the use of the platelet rich plasma analyzing system to determine the quality and likely performance and effectiveness of the derived PRP. FIG. 3 depicts a home screen 26a that includes electronic baseline and PRP buttons 50, 52. Home screen 26a also includes an electronic archive button 54 that allows the user to access other baseline tests for the whole blood sample.

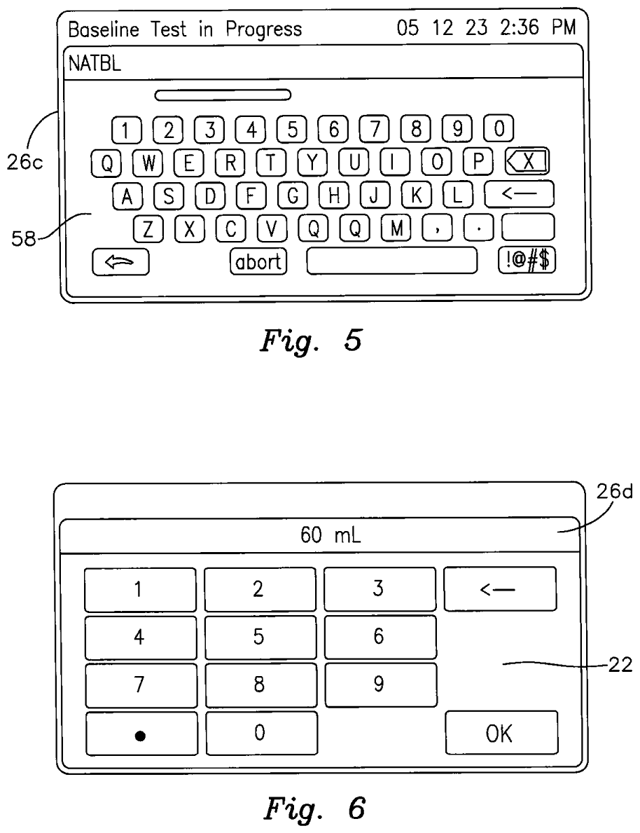

The user collects and prepares a whole blood sample, typically using a 60 ml or alternatively sized syringe. A small amount of that sample is then prepared and introduced into the analyzer, which again may comprise the Emcyte Regenistat™ brand analyzer or alternative devices. Baseline key 50 is actuated to start the test. As shown by screen 26b and 26c in FIGS. 4 and 5, while the test is being conducted, the user may identify the sample using inputs comprising either a barcode 56 or an electronic keyboard 58. In the example described herein, the baseline sample is identified as “NATBL”. As the analyzer continues to run the test on baseline sample NATBL, the user employs the user interface 22 on screen display 26d to enter the volume of the whole blood sample in the syringe. In this example, the volume is 60 ml.

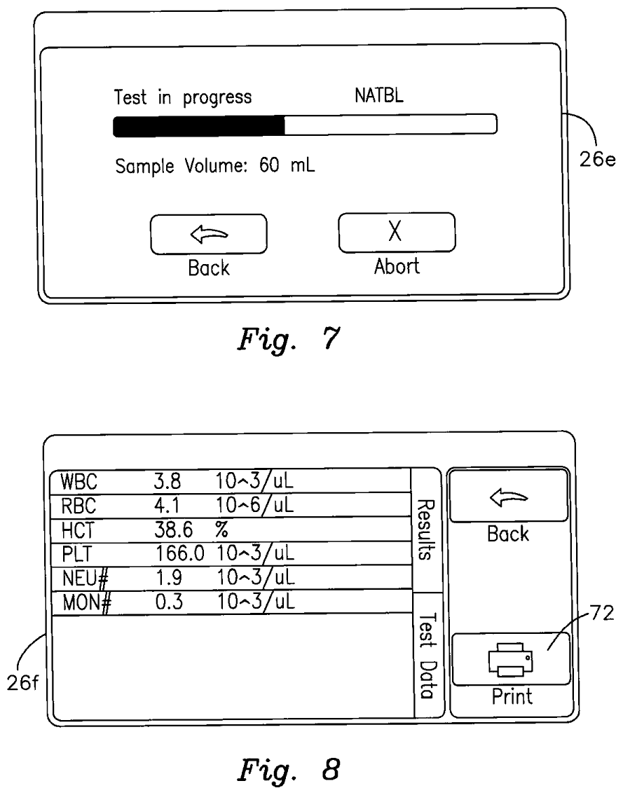

FIG. 7 illustrates screen display 26e revealing that the test is in progress. The baseline specimen ID and sample volume are reflected. Electronic buttons are provided to go back to a prior screen or to abort the test entirely.

When the baseline CBC test is completed, screen display 26 indicates the baseline test results for specimen NATBL. Specifically, screen display 26f, FIG. 8, indicates the levels of white blood cells (WBC), red blood cells (RBC), platelets (PLT), neutrophils (NEU), and monocytes (MON). Hematocrit, or the percentage of red blood cells in the sample, is also listed. Other constituent components of the blood sample such as hemoglobin (not shown), may also be displayed. These results can be printed using printer button 72.

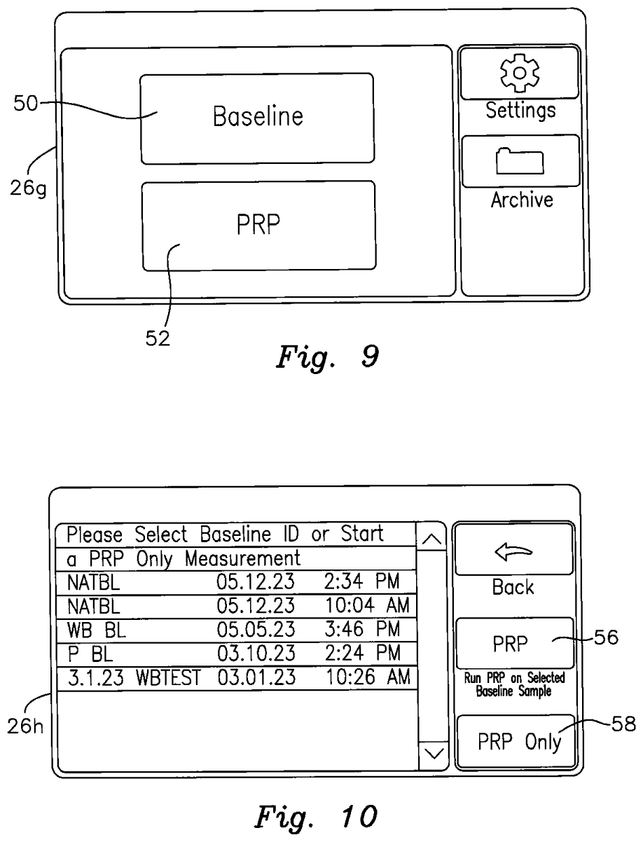

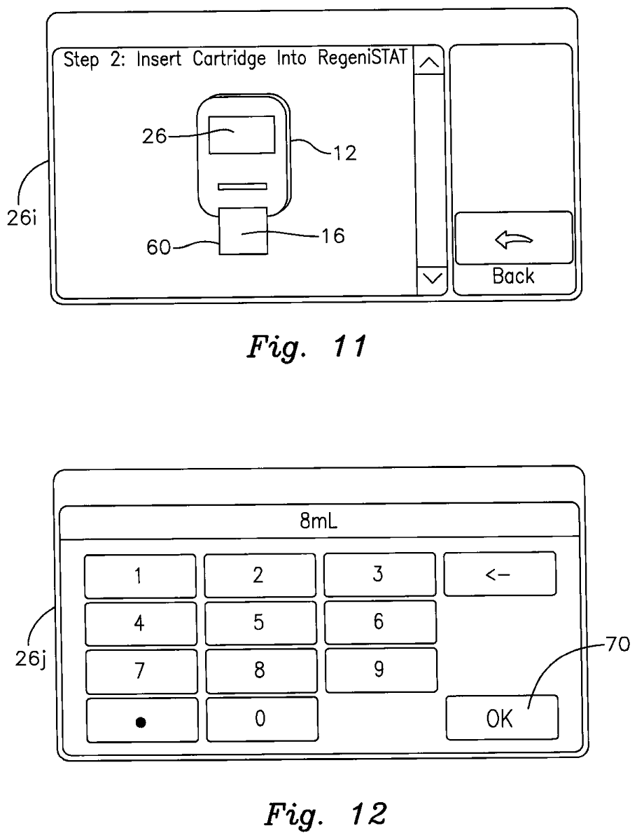

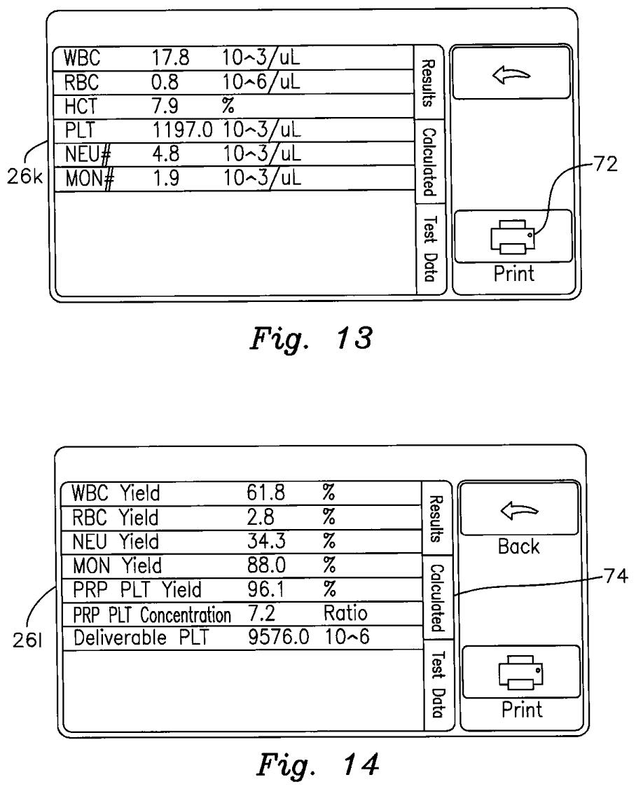

As indicated in FIG. 9, the user next presses the electronic button 52 on screen 26g to commence the PRP test. Typically, the platelet rich plasma has been prepared from whole blood sample using available PRP-producing equipment and techniques, as previously described. When the PRP test is commenced, screen 26h, FIG. 10, is revealed, which lists previously archived CBC baseline tests including the corresponding test “NATBL”. To compare the comparative PRP test in accordance with this invention, the user selects the archived test NATBL and presses the electronic button PRP button 56. The user may optionally choose to perform a PRP only count and to do this the user presses button 58 on screen 26h.
To perform the comparative test that yields PRP performance and quality data, the user presses electronic button 56. As indicated in FIG. 11, screen 26i graphically instructs the user to insert a cartridge 60 bearing a small amount of derived PRP 16 into the test port of analyzer apparatus 12. When the PRP is introduced into the analyzer, a screen display 26j, FIG. 12, appears. That screen includes the operator interface that allows the volume (e.g., 8 ml) of the derived PRP from which the test specimen was taken to be input into the analyzer. The input volume is then used along with the baseline sample volume (e.g., 60 ml) in the comparative calculations so that the PRP quality and performance data is determined. In this example, an 8 ml sample of PRP has been derived from the whole blood sample.

As further shown in FIG. 12 after the PRP volume is entered, the operator presses an “OK” button 70. The PRP count is then displayed on screen 26k, as shown in FIG. 13. This reveals that the desired constituent components of the PRP, namely white blood cells, platelets, neutrophils and monocytes have all increased significantly from the levels contained in the baseline CBC, FIG. 8. The PRP results can then be printed using a printer button 72.

To date, prior art analyzers have only displayed results for the whole blood sample and PRP in the respective manners shown in FIGS. 8 and 13. No prior analyzers have comprehensively compared the two results to provide an indication of the quality and performance of the derived PRP. Rather, physicians and other medical professionals have had to perform tedious calculations to arrive at such quality and performance data.

The present system goes beyond the prior art by calculating and displaying performance data that has yet not been automatically determined by any existing analyzer. Specifically, PRP performance calculator 25, FIGS. 1 and 2, is programmed to produce such results. In particular, as shown by screen 26i, FIG. 14, when the calculator tab 74 is pressed, the PRP results shown in FIG. 13 are compared against the baseline CBC results shown in FIG. 8. PRP quality and performance calculations are performed by factoring in the quantitative results shown in FIGS. 8 and 13 and the whole blood sample and PRP volumes, FIGS. 6 and 12. In particular, screen 26i reveals that the PRP yields 61.8% of the white blood cells present in the baseline sample. The PRP further yields a 34% neutrophil yield and an 88% monocyte yield. Quite significantly, the PRP exhibits a 96.1% yield of valuable and medically beneficial PRP platelets. The PRP exhibits a platelet concentration that is 7.2 times that of the platelet concentration of the baseline CBC. Calculator 25 also determines that 9576.0×10⁶ deliverable platelets were available in the derived PRP. FIG. 14 also illustrates that the red blood cell yield from the PRP has decreased significantly (i.e., from 38.6% as indicated by the hematocrit (HCT) level in FIG. 8 to 2.8% RBC yield in FIG. 14. All of these measured quantities provide the physician with an immediate, accurate and reliable measure of the quality and performance of the tested PRP. Medical professionals do not have to perform tedious, laborious and time-consuming independent calculations that involve the converting milliliters to microliters and vice versa. The user likewise avoids having to perform independent exponential calculations. The analyzer is programmed to determine the PRP's qualitative and performance data integrally which facilitates that determination considerably.

As previously indicated, calculator 25 can also be programmed to automatically calculate the number of platelets that will be delivered in a patient injection dosage of a selected dosage. For example, referring to FIG. 15, the user engages the “Test Data” tab 79 on screen 26m. Initially a test dosage volume screen (not shown) analogous to the volume screens shown in FIGS. 6 and 12 appears. This allows the user to enter a selected injection dosage. If the selected injection dosage is 2 ml, that amount is reflected on screen 26m at 80. The previously entered PRP volume (8 ml) is also indicated at 82. Calculator 25 is programmed to compute the injection dosage number of platelets as 2394.0×10⁶, which is ¼ the number of deliverable platelets in the derived PRP. This is the same proportion as the volume of the injection dosage is to the volume of the PRP derived from the whole blood sample. This feature permits the physician or other user to quickly, conveniently and effectively assess the efficacy of an injection dosage without requiring undue independent calculation. In alternative embodiments, the calculation of the quantity of platelets or other PRP constituents in a selected injection dosage can be performed in a microprocessor or other electronic circuitry separate from calculator 25.

The present invention provides an improved measure of the quality, performance and effectiveness of a derived PRP product by automatically calculating and displaying metrics that are not readily apparent from the simple baseline CBC and derived PRP blood counts alone. The system disclosed herein is constructed and programmed to calculate and display metrics such as constituent component yields, platelet concentration, and number of deliverable platelets that indicate to the medical professional the quality and likely performance and medical effectiveness of a derived PRP product in a more immediate, reliable, understandable and compelling manner that than has heretofore been provided. This likewise immediately illustrates the effectiveness of the PRP process involved. More specifically, the analyzer automatically and accurately measures and displays the amount of constituent(s) (eg platelets) contained in selected injection dosages of PRP derived from a baseline whole blood sample. This enables the physician to better assess treatment efficacy relative to treatment dose.

Accordingly, the present invention provides for a system that is integrated into a hematology or blood analyzer in order to quickly and conveniently provide a reliable, understandable qualitative measure and indication of the effectiveness of a particular PRP product derived from a whole blood sample. While this detailed description has set forth particularly preferred embodiments of this invention, numerous modifications and variations of the composition, elements, and steps of this invention, all within the scope of the invention, will readily occur to those skilled in the art. Accordingly, it is understood that this description is illustrative only of the principles of the invention and is not limitative thereof.

Although specific features of the invention are shown in some of the drawings and not others, this is for convenience only, as each feature may be combined with any and all of the other features in accordance with this invention.

1. A system for analyzing the performance and quality data of a constituent element in a platelet rich plasma (PRP) derived from a whole blood sample, said system comprising:
means for electronically determining a complete blood count (CBC) for the whole blood sample and a PRP count for the derived PRP;
electronic calculator means responsive to input signals received from said means for determining, said calculator means for comparing said determined CBC and PRP counts and calculating for the constituent element performance and quality data from the group including yield, concentration and deliverable quantity of the constituent element in the derived PRP; and
means for displaying said calculated performance and quality data of the constituent element in the derived PRP.

2. The system of claim 1 further including means for inputting the volumes of the whole blood sample and derived PRP into at least one of said means for determining and said calculator means.

3. The system of claim 1 in which said means for displaying include a user interactive screen operatively interconnected to a signal output of said calculator for selectively displaying said calculated performance and quality data and said determined complete blood and PRP counts.

4. The system of claim 2 in which said means for inputting include a user Interactive screen.

5. A method of analyzing the performance and quality data of a constituent element in platelet rich plasma (PRP) derived from a whole blood sample, said method comprising:
providing a blood analyzer that includes electronic means for determining a complete blood count (CBC) for the whole blood sample and a PRP count for the derived PRP, which analyzer further includes electronic calculator means for comparing the determined CBC and PRP counts and calculating for the constituent element performance and quality data from a group including yield, concentration and deliverable quantity of the constituent element in the derived PRP and further providing said analyzer with an interactive user interface and an electronic display;
introducing the whole blood sample into the analyzer;
inputting into the analyzer the volume of the whole blood sample;
operating the means for determining to generate a CBC count for the whole blood sample;
deriving PRP from the whole blood sample;
introducing the derived PRP into the analyzer;
inputting into the analyzer the volume of the derived PRP;
operating the means for determining to generale a PRP count for the derived PRP:
operating the calculator means to compare the CBC and PRP counts and calculate for the constituent element performance and quality data from the group including percentage yield, concentration and deliverable quantity of the constituent element in the derived PRP; and
displaying on the display screen the calculated performance and quality data for the constituent element in the derived PRP.

6. The method of claim 5 further including the step of displaying at least one of the determined CBC and PRP blood counts on the display screen.

7. The method of claim 5 in which the electronic means for determining and electronic means for calculating are operated through the interactive user interface.

8. The method of claim 5 in which the volumes of whole blood sample and derived PRP are input using the interactive user interface.

9. The method of claim 5 in which the interactive user interface is included on the display screen.

10. The system of claim 1 further including an archive database for storing multiple CBC counts and corresponding PRP counts measured by said means for determining.

11. The system of claim 10 further including a user interactive screen that allows an archived CBC count and corresponding PRP count to be selected and input to the calculator means for comparison and calculation of performance and quality data of the derived PRP from which the archived PRP count was determined.

12. The system of claim 1 further including a user interactive interface that is selectively engaged to operate the means for determining and calculator means.

13. The system of claim 12 in which said means for displaying include a user interactive screen and said user interactive interface include electronic keys integrated into said user interactive screen.

14. The system of claim 1 further including means for determining a deliverable quantity of a constituent element of the derived PRP contained in a selected injection dosage having a volume that differs from said input volume of the derived PRP.
